(19) United States
(12) Patent Application Publication
(10) Pub. No.: US 2009/0041874 A1
(43) Pub. Date: Feb. 12, 2009

(54) Karaya Gum-Based Hydrophilic Gel System for Skin Care

(75) Inventors: Frank Theobald, Bad Breisig (DE); Wolfgang Laux, Diez (DE); Rene Eifler, Mulheim-Karlich (DE)

Correspondence Address: D Peter Hochberg Co;The Baker Building, Sixth Floor, 1940 East 6th Street, Cleveland, OH, 44114-2294, US

(73) Assignee: LTS LOHMANN THERAPIE-SYSTEME AG, Andernach (DE)

(21) Appl. No.: 11/883,686

(22) PCT Filed: Jan. 28, 2006
(86) PCT No.: PCT/EP06/00748

§ 371 Date: Aug. 3, 2007

(30) Foreign Application Priority Data
Feb. 7, 2005 (DE) 10 2005 005 573.7

(51) Int. Cl. A61K 36/886; A61K 31/355; A61K 31/34; A61K 31/16; A61K 31/045; A61K 31/352; A61K 31/05; A61K 31/125; A61K 36/15; A61P 17/16

(52) U.S. Cl. 424/744; 514/458; 514/474; 514/625; 514/724; 514/453; 514/731; 514/688; 424/770

(57) ABSTRACT

A hydrophile gel system for cosmetic and/or skin care applications. The gel system comprises a detachable carrier film and a hydrogel which contains at least 15 percent by weight of karaya gum and whose water content is less than 5 percent by weight.

### CROSS-REFERENCE TO RELATED APPLICATIONS

This application is a National Stage application of International Application No. PCT/EP2006/000748, filed on Jan. 28, 2006, which claims priority of German application number 10 2005 005 573.7, filed on Feb. 7, 2005.

### BACKGROUND OF THE INVENTION

1. Field of the Invention

The present invention relates to skin care creams. More particularly, the present invention relates to a hydrophile gel system for cosmetic and/or skin care applications.

2. Description of the Prior Art

Skin care creams used in the field of cosmetics frequently contain hydrogels. These creams are very popular with the consumer because they have a pleasant, cooling effect. However, hydrogel-containing skin care creams are felt to have the disadvantage that these semi-solid preparations always leave a film on the skin which can stick to textiles or be inadvertently smeared.

### SUMMARY OF THE PRESENT INVENTION

It was thus the object of the present invention to provide a product for topical application in the cosmetics field which comprises a hydrogel that does not leave a moist film on the skin.

Another disadvantage of hydrogels per se is that due to their absorbing water from the skin due to “Perspiratio insensibilis”, which term refers to the imperceptible evaporation of water via the skin, they will swell increasingly and thereby lose their cohesion and start to flow.

It was therefore a further object of the present invention to provide a product which comprises a hydrogel that, if applied topically, does not lead to an unacceptable impairment of cohesion, even if the product is applied over a prolonged period of time.

The object is achieved with a hydrophile gel-system comprising a detachable carrier film and a hydrogel. The hydrogel contains at least 15 percent by weight of karaya gum and has a water content of less than 5 percent by weight, preferably less than 1 percent by weight.

As a rule, polymers that are required for forming a hydrogel can be processed in aqueous media only at relatively low concentrations of approximately 1 to 5 percent by weight. Higher amounts of a corresponding polymer will result in masses that are no longer spreadable, so that it is not possible to coat a carrier film with a mass that contains more than 5 percent by weight of a polymer required for forming the hydrogel as the matrix since such a mass will not possess sufficient flowability.

Surprisingly, it has emerged that it is possible to process masses containing markedly higher amounts of karaya gum if formulations are used wherein the water-content is kept low. Swelling of the karaya gum can thereby be prevented. Thus, it was found that hydrogel matrices having a content of up to 40 percent by weight of karaya gum can be manufactured in the presence of less than 5 percent by weight of water, preferably less than 1 percent by weight of water.

The term “karaya gum”, also called Sterculia gum, refers to the dried exudate of trees, native to Africa and India, of the family of the Sterculiaceae. It is a polysaccharide which is based on galactose, rhamnose, galacturonic acid and glucuronic acid and which in cold water swells to 60-100 times its volume but is insoluble therein. Karaya gum is a good film-forming agent and has good wet-adhesive strength. It forms seemingly homogeneous dispersions with water which, at concentrations of greater than 3 percent by weight, no longer flows.

An advantage of using hydrogels having a content of greater than or equal to fifteen percent by weight of karaya gum is that, even after absorbing very large amounts of water, they retain their consistency and do not start to flow. For that reason, the cohesion of the hydrogel is maintained over the entire application period of the gel system according to the invention, and the gel system can be removed without leaving residues on the skin, even after application of several days.

### DETAILED DESCRIPTION OF THE PRESENT INVENTION

In a preferred embodiment, the gel system according to the present invention has a pressure-sensitive adhesive hydrogel matrix based on karaya gum, wherein the hydrogel contains, in addition to the karaya gum, at least one pressure-sensitive adhesive polymer to improve the self-adhesive properties of the gel system.

The pressure-sensitive adhesive polymer is preferably selected from the group comprising polyacrylates, polydimethyl siloxanes, polyisobutenes, polyisobutylenes, styrene-isoprene-styrene block copolymers, resins, and combinations of these polymers.

The portion of pressure-sensitive adhesive polymer in the hydrogel is 0.5 to 80 percent by weight, preferably 5 to 60 percent by weight, more preferably 15 to 50 percent by weight, and especially preferably 30 to 40 percent by weight.

The hydrogel matrix is located on a suitable carrier film, which is peeled off before applying the system to the skin.

In its simplest embodiment, an additional cover film on that side of the hydrogel which is opposite the detachable carrier film can be dispensed with if the matrix is adjusted so as to be appropriately “dry” and slightly adhesive and if the hydrogel is of a cuttable consistency. The term “dry” matrix in this context means a hydrogel wherein the portion of karaya gum is at least 20 percent by weight and whose water portion is less than 5 percent by weight.

On the other hand, it is possible for the hydrogel matrix to be covered, on the side opposite the carrier film, with an additional film. In that case, the additional cover film may be water vapour-pervious or water-vapour impervious.

It has, surprisingly, been found that skin care substances, too, can be incorporated in karaya gum-based hydrogels. Incorporating skin care substances is advantageous in hydrogels for cosmetic and/or skin care applications in order to prevent the skin from drying as a consequence of water loss. There is, however, a danger of phase separation occurring since the, generally lipophile, substances intended for skin care are not compatible with the hydrophile karaya gum.

Surprisingly, incorporation of at least one moisture-absorbing or of at least one emulsifying substance into the hydrogel containing at least 15% by weight of karaya gum enables the incorporation of skin care substances, especially of lipophile substances, into the hydrogel without the occurrence of undesirable phase separation. The absorbing or emulsifying substances that enable incorporation of skin care substances into the karaya gum-based hydrogel according to the invention are selected from the group comprising cyclodextrins and their derivatives; silicic acid and its derivatives, medicinal charcoal, emulsifiers and complex emulsifiers.

Preferred cyclodextrin derivatives are selected from the group comprising β-hydroxypropyl-cyclodextrin, methyl-β-cyclodextrin, hydroxypropyl-γ-cyclodextrin and hydroxypropyl-α-cyclodextrin.

Preferred derivatives of silicic acid are selected from the group comprising highly dispersed silicon dioxide and other silicates.

Preferred emulsifiers are selected from the group comprising sodium palmitate, sodium stearate, triethanolamine stearate, sodium lauryl sulfate, sodium cetyl sulfonate, sodium glycocholate, gum arabic, alkonium bromide, benzalkonium bromide, cetylpyridinium chloride, cetyl alcohol, stearyl alcohol, higher linear fatty alcohols with 6 to 22 carbon atoms preferably obtained by reduction of fats and fatty acids, partial fatty acids of polyhydric alcohols, partial fatty acid esters of sorbitan, partial fatty acid esters of polyoxyethylene, fatty alcohol ethers of polyoxyethylene, fatty acid esters of Saccharose, fatty acid esters of polyglycerols, and lecithin.

As the preferred complex emulsifier, cetyl stearyl alcohol is used.

Preferably the portion of absorbing and/or emulsifying substances in the hydrogel matrix is 0.5 to 25 percent by weight.

The skin care substance that may be contained in the hydrogel matrix is preferably selected from the group comprising aloe vera, vitamin E, vitamin C, dexpanthenol, glycerol, propylene glycol, eucalyptol, menthol, camphor, pine needle oil, cineol, borneol, and bisabolol.

The portion of skin care substance(s) in the hydrogel preferably amounts to 1 to 50 percent by weight.

To produce the hydrogel matrix according to the invention, in order to prevent the swelling of the karaya gum there is no water used as an agent for dissolving or emulsifying the ingredients. Instead, organic solvents are used. Suitable organic solvents may be selected from the group consisting of methanol, ethanol, isopropanol, ethyl acetate, n-hexane, heptane, and cyclohexane.

Preferred formulations of the hydrogel according to the present invention are:

### EXAMPLE 1

| | |
|---|---|
| Durotak 387-2054 | 36.2% |
| Al-acetyl acetonate | 0.5% |
| Karaya gum | 36.7% |
| [[Tween]] TWEEN ® 80 | 6.9% |
| [[Atmos]] ATMOS ® 300 | 6.9% |
| Camphor | 6.2% |
| Menthol | 2.9% |
| Pine needle oil | 3.7% |

### EXAMPLE 2

| | |
|---|---|
| Karaya gum | 19.00% |
| Glycerol (anhydrous) | 29.00% |
| Propylene glycol | 19.50% |
| Silicic acid | 7.00% |
| Hydroxypropyl-β-cyclodextrin | 6.50% |
| Menthol | 3.45% |
| Pine needle oil | 3.80% |
| Camphor | 4.75% |
| Durotak 387-2287 | 7.00% |

All percentages are percent by weight.

### EXAMPLE 3

To prepare a gel system of the formulation according to Example 2, Durotak 387-2287 were provided. Glycerol, propylene glycol and pine needle oil were weighed in and homogenised at medium stirring rate. Thereafter, camphor and menthol were successively weighed in, added, in both cases while stirring, and dissolved. Addition of menthol resulted in an opaque solution, into which, successively, hydroxypropyl-β-cyclodextrin and silicic acid (SIDENT® 22 S) were weighed in, added while stirring, and stirred until the mass was homogeneous. Stirring was continued until there was no longer any change in the viscosity of the mass. The mass was cooled in an ice bath and stirring was continued at the lowest stirring level. Then, karaya gum was added while stirring, and homogenised, the stirrer being left at the lowest stirring level, and cooling of the mass was continued without interruption.

The resulting mass was coated on a nonwoven (VILMED® M1585 x/Hy) and dried. Finally, a one-side siliconised paper was laminated onto the hydrogel matrix as a carrier film.

The gel system according to the invention is suited, in particular, for topical application in the fields of cosmetics and/or skin care.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

We claim:

1. A hydrophile gel system for cosmetic and/or skin care applications, said hydrophile gel system comprising a detachable carrier film and a hydrogel matrix, wherein said hydrogel matrix contains at least 15 percent by weight of karaya gum, at least one skin care substance, and at least one moisture-absorbing or emulsifying substance which is selected from the group consisting of cyclodextrins, cyclodextrin derivatives, silicic acid, silicic acid derivatives, medicinal charcoal, emulsifiers and complex emulsifiers, and wherein said hydrogel has a water content of less than 1 percent by weight.

2. The gel system according to claim 1, wherein the hydrogel matrix contains at least one pressure-sensitive adhesive polymer which is selected from the group of polymers consisting of polyacrylates, polydimethyl siloxanes, polyisobutenes, polyisobutylenes, styrene-isoprene-styrene block copolymers, resins, and combinations of these polymers.

3. The gel system according to claim 1, wherein the portion of pressure-sensitive adhesive polymer in the hydrogel is 0.5 to 80 percent by weight.

4. The gel system according to claim 1, wherein a film selected from the group consisting of a water-vapour permeable film and a water-vapour impermeable film covers the hydrogel matrix on the side opposite the carrier film.

5. The gel system according to claim 1, wherein the cyclodextrin derivative is selected from the group consisting of β-hydroxypropyl-cyclodextrin, methyl-β-cyclodextrin, hydroxypropyl-γ-cyclodextrin and hydroxypropyl-α-cyclodextrin.

6. The gel system according to claim 1, wherein the silicic acid derivative is a highly dispersed silicon dioxide.

7. The gel system according to claim 1, wherein the emulsifier is selected from the group consisting of sodium palmitate, sodium stearate, triethanolamine stearate, sodium lauryl sulfate, sodium cetyl sulfonate, sodium glycocholate, gum arabic, alkonium bromide, benzalkonium bromide, cetylpyridinium chloride, cetyl alcohol, stearyl alcohol, higher linear fatty alcohols with 6 to 22 carbon atoms, partial fatty acids of polyhydric alcohols, partial fatty acid esters of sorbitan, partial fatty acid esters of polyoxyethylene, fatty alcohol ethers of polyoxyethylene, fatty acid esters of saccharose, fatty acid esters of polyglycerols and lecithin.

8. The gel system according to claim 1, wherein the complex emulsifier is cetyl stearyl alcohol.

9. The gel system according to claim 1, wherein the portion of absorbing and/or emulsifying substance in the hydrogel matrix is 0.5 to 25 percent by weight.

10. The gel system according to claim 1, wherein the skin care substance is selected from the group consisting of aloe vera, vitamin E, vitamin C, dexpanthenol, glycerol, propylene glycol, eucalyptol, menthol, camphor, pine needle oil, cineol, borneol and bisabolol.

11. The gel system according to claim 1, wherein the portion of skin care substance in the hydrogel matrix is 1 to 50 percent by weight.

12. Use of a gel system according to claim 1 for topical application in the cosmetics and/or skin care fields.

13. A process for manufacturing gel system according to claim 1, comprising the step of using an organic solvent to dissolve and/or emulsify the ingredients, wherein said organic solvent is selected from the group consisting of methanol, ethanol, isopropanol, propanediol, ethyl acetate, n-hexane, heptane and cyclohexane.

14. The gel system according to claim 3, wherein the portion of pressure-sensitive adhesive polymer in the hydrogel matrix is 5 to 60 percent by weight.

15. The gel system according to claim 14, wherein the portion of pressure-sensitive adhesive polymer in the hydrogel matrix is 15 to 50 percent by weight.

16. The gel system according to claim 15, wherein the portion of pressure-sensitive adhesive polymer in the hydrogel matrix is 30 to 40 percent by weight.
